(19) United States
(12) Patent Application Publication
(10) Pub. No.: US 2014/0100181 A1
(43) Pub. Date: Apr. 10, 2014

(54) TOPICAL PHARMACEUTICAL COMPOSITION CONTAINING A WATER-SENSITIVE ACTIVE PRINCIPLE

(71) Applicant: Galderma S.A., Cham (CH)

(72) Inventors: Karine NADAU-FOURCADE, Villeneuve Loubet (FR); Nathalie Barthez, Saint Laurent du Var (FR); Laetitia Mazeau, Cagnes sur Mer (FR)

(73) Assignee: Galderma S.A., Cham (CH)

(21) Appl. No.: 14/101,055

(22) Filed: Dec. 9, 2013

Related U.S. Application Data
(62) Division of application No. 13/141,785, filed on Sep. 20, 2011.
(60) Provisional application No. 61/193,793, filed on Dec. 23, 2008.

(51) Int. Cl. A61K 31/7048; A61K 31/05

(52) U.S. Cl. CPC A61K 31/7048; A61K 31/05
USPC 514/30

(57) ABSTRACT

A topical pharmaceutical composition including, as a pharmaceutical active agent, a water-sensitive compound in a solubilised form in a physiologically acceptable medium is described. A method for preparing such a composition, and uses thereof in dermatology are also described.

The invention relates to a topical pharmaceutical composition comprising as pharmaceutical active agent a water-sensitive compound, in a dissolved form, in a physiologically acceptable medium, to the process for preparing it and to its dermatological use.

In the field of dermatology and of the formulation of pharmaceutical compositions, a person skilled in the art is led to use compositions that must be physically and chemically stable. They must also allow the release of the active agent and promote its penetration into the skin layers in order to improve its efficacy.

Many active agents have the difficulty of being very sparingly soluble in the cosmetic or pharmaceutical solvents commonly used, especially water, and/or of being sensitive to an aqueous, oxidizing environment. This water sensitivity may lead to chemical instability of the active agent and/or to crystallization of the initially dissolved active agent. This water sensitivity thus limits their formulation in topically applied cosmetic or dermatological compositions.

To obtain physical and chemical stability of a composition comprising a water-sensitive active agent, a person skilled in the art is tempted to use anhydrous compositions or compositions with a very high fatty phase content. This therefore gives the composition a greasy and occasionally tacky appearance, resulting in poor cosmetic acceptability. A person skilled in the art also knows that failure to adhere to the prescribed treatment for the reasons mentioned previously is one of the main causes of failure; in particular, for the treatment of psoriasis, the article “Patients with psoriasis and their compliance with medication” (Richards at al., J. Am. Acad. Dermatol. October 99, pp. 581-583) indicates that close to 40% of patients with a chronic disease such as psoriasis do not follow their treatment. It has been demonstrated that a patient's adherence to his treatment is directly linked to the characteristics of the vehicle of the applied composition. The article “Patients with psoriasis prefer solution and foam vehicles: a quantitative assessment of vehicle preference” (Housman et al.; CUTIS, December 2002 vol. 70, pp. 327 to 332) indicates that psoriatic patients prefer a solution, a foam or a light emulsion, rather than an ointment, or a thick, greasy cream.

Specifically, on reading the prior art, the existing compositions, which allow a water-sensitive active agent to be formulated in dissolved form, often contain a high percentage of oil and often of petroleum jelly to promote the occlusiveness and the penetration of the active agent. They therefore have the drawback of being very greasy and tacky, and thus of not promoting comfort and ease of application. The other types of composition commonly encountered in the prior art contain a high percentage of penetration-enhancing glycol in order to promote the penetration of the active agent, but are often tacky and may cause intolerance problems. (“The critical role of the vehicle to therapeutic efficacy and patient compliance” Piacquadio et al, Journal of American Academy of Dermatology, August 1998).

In order to overcome this problem of discomfort and of ease of application, a person skilled in the art is thus seeking to formulate the active agent in a vehicle that is cosmetically acceptable as well as being pharmaceutically effective. This is generally the case for emulsions, containing an aqueous phase. The main problem to solve is thus that of stabilizing the water-sensitive active agent and the composition despite the presence of water in the composition.

The phenomena of chemical degradation and/or crystallization of the active agent in the presence of water have the consequence of reducing or losing efficacy and of uncertainty as regards the dose of active agent implemented during its use, which runs counter to the desired objective. In addition, this degradation of the active agent and/or its crystallization can modify the overall stability of the compositions and also their appearance.

According to the invention, the term “active agent that is sensitive in the presence of water” thus means a chemically and/or physically unstable active agent. The term “chemical instability” especially means degradation of the active principle. The term “physical instability” especially means crystallization or precipitation of the active agent, or modification of its colour within the composition.

A physically stable composition according to the invention is consequently a composition that does not present any macroscopic change of appearance (phase separation, change of aspect colour, etc.) or microscopic change of appearance (recrystallization of the active agents) after storage at temperatures of 25° C., 4° C. and 40° C., for 2, 4, 8 or 12 weeks.

A chemically stable composition according to the invention is, consequently, a composition in which the content of active principle remains stable after three months at room temperature and at 40° C. A stable content of active principle means according to the invention that the content shows very little variation relative to the initial content, i.e. that the variation in the content of active principle at time T should not be less than 90% and more particularly than 95% of the initial content at T0.

There is thus a need for a composition that can satisfy one or more of the following aspects: provide good stability of the formulation under cold and warm conditions, in particular as regards maintenance of the globule size and the absence of phase separation, show good resistance of the active agent to oxidation phenomena, allow good chemical stability of the active agent and good availability thereof for the skin. It is also useful to be able to provide a composition that permits a high dispersed volume fraction. It is moreover useful for the mode of preparation of such compositions to be easy and advantageous.

A person skilled in the art is thus seeking to improve these parameters by the present invention.

Other parameters should also be taken into account by a person skilled in the art for the choice of ingredients of a pharmaceutical composition. Specifically, the pharmaceutical composition that may be used according to the invention as medicament will also have to be formulated in accordance with the pathology to be treated.

By way of non-limiting example, a composition for treating acne will need to be of non-greasy cosmetic appearance, whereas a composition for treating atopic dermatitis will need to be emollient and moisturizing, and may be richer in fatty substances, while at the same time avoiding the non-cosmetic greasy appearance.

The problem that the present invention proposes to solve herein is thus that of designing an aqueous pharmaceutical composition of oil-in-water emulsion type, which is physically and chemically stable, comprising at least one water-sensitive active principle, in dissolved form. The said composition must be other than an ointment. The composition according to the invention may thus be in various emulsion forms: sprayable emulsion, lotion, milk, thick cream of more or less rich variable texture according to the pathology to be treated. It must also be easy to use and of acceptable cosmeticity for application to any area of the body that may be affected by the pathology.

In the present invention, the Applicant has shown, surprisingly, that it is possible to obtain a composition of oil-in-water emulsion type, containing a water-sensitive active agent, dissolved in the inner fatty phase, which is physically and chemically stable, the said composition comprising:
at least one water-sensitive active principle,
a fatty phase containing at least one lipophilic phase that is a solvent for the active agent,
at least one polyol,
at least 5% water,
   characterized in that it is topical and that it comprises at least one surfactant of the family of sucroesters or polyglycerol esters.

The term “dissolved form of the active agent” means a dispersion of the active agent in molecular form in a liquid, no crystallization of the active agent being visible to the naked eye or even under a cross-polarized optical microscope.

In one preferred embodiment according to the invention, the composition comprises at least one water-sensitive active agent chosen from liposoluble vitamin derivatives such as those of vitamin A (retinol), E or C and more particularly those of vitamin D, macrocyclic lactones or phenolic derivatives.

Vitamin D derivatives show known instability in media containing water and more particularly at acidic pH values. These derivatives are usually formulated in anhydrous fatty preparations.

Similarly, phenolic derivatives show very rapid oxidation in aqueous media, even in the presence of antioxidants. It is thus difficult to formulate them in the presence of water.

Macrocyclic lactones also have known instability in aqueous media, which makes it difficult to formulate them in an oil-in-water emulsion.

These active principles all have the drawback of being unstable in aqueous media, and/or sensitive to acidic pH and thus difficult to formulate in a composition containing water. Even when dissolved in the oily phase of a standard oil-in-water emulsion, these active principles may migrate into the aqueous phase, thus chemically and/or physically destabilizing the composition.

The composition according to the invention thus comprises at least one water-sensitive active principle chosen from vitamin D derivatives, macrocyclic lactones and a phenolic derivative.

As vitamin D derivatives that may be used according to the invention, mention may be made of the compounds chosen from calcitriol, calcipotriol and 4-[6-ethyl-4′-(1-ethyl-1-hydroxypropyl)-2′-propylbiphenyl-3-yloxymethyl]-2-hydroxymethylphenylmethanol. Calcitriol will preferably be used.

The composition according to the invention comprises between 0.00001% and 0.1% of at least one vitamin D derivative by weight relative to the total weight of the composition, and preferably from 0.0001% to 0.1%. Preferentially, the composition according to the invention contains from 0.0001% to 0.05% and preferably from 0.003% to 0.06% of calcitriol.

As macrocyclic lactones that may be used according to the invention, mention may be made of compounds chosen from avermectins, such as ivermectin, invermectin, avermectin, abamectin, doramectin, eprinomectin and selamectin, aversectin B, AB or C, emamectin B1a, emamectin B1b and derivatives thereof, or latidectin. The compound of the avermectin family is preferably ivermectin.

In the compositions according to the invention, the compound of the avermectin family is present in a concentration of between 0.001% and 10% by weight and preferably between 0.01% and 5% by weight relative to the total weight of the composition comprising it. In particular, the composition comprises 0.75%, 1%, 1.5% or 2% ivermectin.

Phenolic derivatives that may be mentioned in a non-limiting manner include hydroquinone, 4-hydroxyanisole, rucinol, hydroquinone monoethyl ether and hydroquinone monobenzyl ether. Preferably, hydroquinone or rucinol is used. Advantageously, the amount of phenolic derivative is from 0.00001% to 10% by weight, preferably from 0.0001% to 6% by weight and more particularly from 0.01% to 5% by weight relative to the total weight of the composition.

The composition according to the invention may also comprise a combination of active agents. The second active agent may be either also water-sensitive or sensitive to acidic pH, and in this case will also be stabilized in the fatty phase. The second active agent may also be an active agent that is compatible with water and/or with an acidic pH and will be introduced into the aqueous phase.

As a non-limiting example of a composition according to the invention comprising a combination of active agents, mention may be made of a combination of a vitamin D derivative stabilized in the fatty phase and of a corticoid stabilized in the aqueous phase. In one preferred embodiment according to the invention, the vitamin D derivative is calcitriol as defined previously. The corticoid preferentially used is Clobetasol propionate, used at between 0.001% and 0.1% and preferably between 0.01% and 0.05% by weight relative to the total weight of the composition.

Mention may also be made of a combination of a phenolic derivative stabilized in the fatty phase and of a retinoid, which is either also stabilized in the fatty phase or dispersed in the aqueous phase. The retinoid dispersed in the aqueous phase may be adapalene and the phenolic derivative dispersed in the aqueous phase may be hydroquinone or rucinol.

The fatty phase according to the invention should be chosen so as to contain at least one solvent phase or solvent oil for the active agent.

The term “solvent oil for the active agent” means an oil in which the active agent is dissolved and chemically stable.

When the active agent of the composition according to the invention is calcitriol, the solvent oil may be, for example, caprylic/capric triglycerides, diisopropyl adipate, octyldodecanol, mineral oil or PPG-15 stearyl ether.

When the active agent of the composition according to the invention is ivermectin, the solvent oil may be, for example, diisopropyl adipate, PPG-15 stearyl ether, octydodecanol, oleyl alcohol, triacetin, caprylic/capric triglycerides, phenoxyethanol or benzyl alcohol.

When the active agent of the composition according to the invention is hydroquinone or rucinol, the solvent oil may be, for example, caprylic/capric triglycerides, diisopropyl adipate, lauroglycol, PPG 15 stearyl ether or castor oil.

Other oils or fatty substances may be added to the solvent fatty phase of the composition in a varied manner by a person skilled in the art in order to prepare a composition having the desired properties, for example in terms of consistency or texture.

Among these oils or fatty substances, mention may be made, in a non-exhaustive manner, of:
plant oils, such as the sweet almond oil sold by Sictia or the sesame oil sold by CPF, palm oil, soybean oil, sesame seed oil, sunflower oil or olive oil,
mineral oils, for instance liquid paraffins of different viscosities, for instance Marcol 152®, Marcol 52® or Primal 352® sold by Esso;
triglycerides, for instance caprylic/capric triglycerides sold under the name Miglyol 812® by Sasol,
esters, for instance octyldodecyl myristate sold under the name MOD by Gattefossé, C12-C15 alkyl benzoate sold under the name Tegosoft TN by Goldschmidt or isononyl isononanoate sold under the name DUB ININ by Stéarinerie Dubois, cetearyl isononanoate sold under the name Cetiol SN by the company Cognis, diisopropyl adipate or Crodamol DA sold by the company Croda, isopropyl palmitate (Crodamol IPP) or isopropyl myristate (Crodamol IPM) sold by the company Croda,
alkoxylated alcohols, for instance POE (15) stearyl ether (Steareth-15), PPG-15 stearyl ether benzoate, PPG-5 Ceteth-20 or POE (20) isohexadecyl ether (Isoceteth-20),
ethers and derivatives, for instance PPG 15 stearyl ether sold under the name Arlamol E® by Croda,
Guerbet alcohols, such as the octyldodecanol sold under the name Eutanol G® by Cognis;
animal oils or substitutes thereof of plant origin when they exist; mention may be made of lanolin, squalene, fish oil, mink oil, with, as a derivative, perhydrosqualene sold under the name Sophiderm® by the company Sophim or under the name Cosbiol® by the company Laserson,
hydrogenated polyisobutenes, for instance Parleam® sold by the company Rossow,
silicone oils, for instance cyclomethicone sold under the name ST-Cyclomethicone 5NF® by Dow Corning or Dimethicone sold under the name Q7 9120 Silicone Fluid® of viscosity 20 cSt to 12 500 cSt sold by Dow Corning, or lipophilic silicone compounds such as polyorganosiloxane elastomers, such as Elastomer 10 sold by Dow Corning,
and mixtures thereof.

The composition according to the invention may also contain fatty substances with a high melting point that are solid at room temperature, or lipophilic gelling agents, also known as lipophilic thickeners.

The term “fatty substances with a high melting point” means compounds chosen from waxes, fatty alcohols, hydrogenated oils and fatty acid esters.

The term “wax” generally means a lipophilic compound, which is solid at room temperature (25° C.), with a reversible solid/liquid change of state, having a melting point of greater than or equal to 30° C., which may be up to 200° C. and especially up to 120° C. As waxes that may be used, mention may be made of carnauba wax, microcrystalline waxes, the beeswax sold under the name Cerabell blanche by Barlocher, or candelilla wax.

As fatty alcohols that may be used, mention may be made of oleyl alcohol, cetyl alcohol, cetearyl alcohol or stearyl alcohol.

The term “hydrogenated oil” means oils obtained by catalytic hydrogenation of animal or plant oils containing linear or branched C₈-C₃₂ fatty chains. Among these, mention may be made especially of hydrogenated jojoba oil, isomerized jojoba oil such as the trans-isomerized partially hydrogenated jojoba oil manufactured or sold by the company Desert Whale under the trade reference iso Jojoba-50®, hydrogenated sunflower oil, hydrogenated castor oil sold especially under the name Cutina HR® by Cognis, hydrogenated coconut oil and hydrogenated lanolin oil.

As fatty acid esters that may be used, mention may be made of lanolin, sold especially under the name Medilan® by Croda, hydrogenated coconut glycerides sold under the name Akosoft 36® by Karlshamns, or diethylene glycol monostearate or propylene glycol monostearate, sold, respectively, under the name Hydrine or Monosterol by Gattefossé or glyceryl behenate sold under the name Compritol 888® by Gattefossé.

Preferably, the fatty phase comprises at least one solvent oil for the active agent, chosen from caprylic/capric triglycerides, mineral or plant oils and esters. Preferentially, the oils according to the invention are Miglyol 812 (caprylic/capric triglycerides), Crodamol DA and liquid paraffin associated with silicone oils such as dimethicone or fatty alcohols.

Thus, the fatty phase of the emulsion according to the invention may be present in a content of between 1% and 95% by weight, preferably between 5% and 85% and more preferentially between 15% and 50% by weight relative to the total weight of the composition.

The chemical and physical stabilities of the composition according to the invention are obtained especially via the choice of the surfactants. Thus, the composition according to the invention also comprises at least one main surfactant chosen from the category of sucrose esters or polyglycerol esters.

Sucrose esters are nonionic surfactants comprising a hydrophilic group formed by the sucrose part and a lipophilic group formed by a fatty acid. As sucrose generally has a total of 8 hydroxyl groups, it is thus possible to obtain sucrose esters ranging from a sucrose “monoester” to a sucrose “octaester”.

Non-limiting examples of sucrose esters that may be mentioned include sucrose stearate, sucrose laurate or sucrose palmitate, sold under the trade name Surfhope by the company Mitsubishi-Kagaku, which are preferred sucrose esters used in the composition according to the invention.

In another embodiment according to the invention, the surfactants that may be used are polyglycerol esters. These are polyglycerolated fatty acid esters obtained by condensation of glycerol. Examples that may be mentioned include decaglyceryl monomyristate and decaglyceryl monolaurate sold under the names S-Face L1001® and S-Face M1001® by the company Sakamoto.

The surfactants according to the invention are used at between 0.01% and 30% by weight, preferentially between 0.1% and 15% and more preferentially between 0.5% and 7% by weight relative to the total weight of the composition.

As the composition according to the invention is an oil-in-water emulsion, it comprises an aqueous phase containing at least 5% and preferably between 5% and 90% water relative to the total weight of the composition.

In one preferred embodiment according to the invention, the aqueous phase also contains a polyol (at the minimum a triol) preferably selected from the group of trihydric alcohols (for instance glycerol), tetrahydric alcohols (for instance diglycerol) and hexahydric alcohols (for instance sorbitol).

Thus, the amount of polyol according to the invention is between 1% and 40% by weight relative to the total weight of the composition.

In one preferred mode, the composition according to the invention contains glycerol in a content of between 1% and 20% and a proportion of water of between 5% and 90%.

In one particularly preferred embodiment, the composition according to the invention also comprises one or more hydrophilic-phase gelling agents. As non limiting examples of gelling agents that may be included in the compositions according to the invention, mention may be made of the Acrylates/C10-30 alkyl acrylate crosspolymer sold under the name Pemulen TR1® or Pemulen TR2® by the company Noveon, the carbomers sold under the name Ultrez 20®, Ultrez 10®, Carbopol 1362® or Carbopol ETD2020NF®, Carbopol 981 or Carbopol 980 by the company Noveon, polysaccharides, non-limiting examples being xanthan gum such as Xantural 180® sold by the company Kelco, gellan gum sold under the name Kelcogel® by the company Kelco, guar gum, cellulose and derivatives thereof such as microcrystalline cellulose and sodium carboxymethyl-cellulose sold under the name Avicel CL-611® by the company FMC Biopolymer, hydroxypropylmethylcellulose, in particular the product sold under the name Methocel E4M premium by the company Dow Chemical, or hydroxyethylcellulose, in particular the product sold under the name Natrosol HHX 250® by the company Aqualon, the family of aluminium magnesium silicates such as Veegum K sold by the company Vanderbilt, the family of acrylic polymers coupled to hydrophobic chains such as PEG-150/decyl/SMDI copolymer sold under the name Aculyn 44® (polycondenate comprising at least, as elements, a polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)), the family of modified starches such as the modified potato starch sold under the name Structure Solanace, or mixtures thereof, and gelling agents of the family of polyacrylamides, such as the mixture Sodium acryloyldimethyltaurate copolymer/isohexadecane/polysorbate 80 sold under the name Sepineo P 600® (or Simulgel 600 PHA®) by the company SEPPIC, the mixture polyacrylamide/isoparaffin C13-14/laureth-7, for instance the product sold under the name Sepigel 305 by the company SEPPIC, the family of carrageenans, in particular divided into four major families: κ, λ, β, ω such as the Viscarin® products and the Gelcarin® products sold by the company IMCD.

Preferred gelling agents that may be mentioned include carbomers, for instance Carbopol 980® or 981®, polyacrylamides, for instance Sepineo P 600® or Simulgel 600 PHA®, and polysaccharides, for instance xanthan gum.

The gelling agent as described above may be used at preferential concentrations ranging from 0.001% to 15% and more preferentially ranging from 0.01% to 5%.

The composition according to the invention may also comprise additives commonly used in pharmaceuticals and cosmetics for giving the said preparation specific properties. A person skilled in the art will adapt the choice of these additives as a function of the expected effect.

Among the additives, examples that may be mentioned include, taken alone or in combination:
antioxidants such as vitamin E and derivatives thereof, for instance DL-α-tocopherol or tocopheryl acetate from Roche; vitamin C and derivatives thereof, for instance Ascorbyl Palmitate from Roche, butylhydroxytoluene sold under the name Nipanox BHT by Clariant, and sodium metabisulfite,
vitamins such as vitamin PP or niacinamide,
calmatives and/or anti-irritant agents such as PPG-12/SMDI copolymer sold by the company Bertek Pharmaceuticals under the trade name Polyolprepolymer-2, or glycyrrhetinic acid or derivatives thereof, for instance Enoxolone sold by the company Cognis, or hyaluronic acid.
lecithins, cholesterol,
preserving agents: Examples of preserving agents that may be mentioned include benzalkonium chloride, bronopol, chlorhexidine, chlorocresol and derivatives thereof, ethyl alcohol, phenoxyethanol, potassium sorbate, diazolidinylurea, benzyl alcohol, parabens, or mixtures thereof, methyl paraben sold under the name Nipagin M by Clariant, and propyl paraben sold under the name Nipasol by Clariant,
acids or bases such as citric acid, sodium citrate, triethanolamine, aminomethylpropanol, sodium hydroxide and diisopropanolamine;
chelating agents.

The additives will be present in the composition according to the invention in proportions ranging from 0 to 20% of the total weight of the composition.

The composition according to the invention thus relates to a topical composition of oil-in-water emulsion type, comprising:
at least one water-sensitive active principle,
a fatty phase containing at least one lipophilic phase that is a solvent for the active agent,
at least one surfactant of the family of sucroesters or polyglycerol esters,
at least one polyol,
at least 5% water.

The composition according to the invention thus relates to a topical composition of oil-in-water emulsion type, comprising:
at least one water-sensitive active principle chosen from vitamins, macrocyclic lactones and phenolic derivatives,
a fatty phase containing at least one lipophilic phase that is a solvent for the active agent,
at least one surfactant of the family of sucroesters or polyglycerol esters,
at least one polyol,
at least 5% water.

According to one preferred embodiment, the composition according to the invention relates to a topical composition of oil-in-water emulsion type comprising:
at least one water-sensitive active principle chosen from vitamin ID derivatives, macrocyclic lactones and phenolic derivatives,
a fatty phase containing at least one lipophilic phase that is a solvent for the active agent,
at least one surfactant of the family of sucroesters or polyglycerol esters,
at least one polyol,
at least one gelling agent,
at least 5% water.

In one particularly preferred embodiment, the composition is in emulsion form and comprises:
between 0.00001% and 0.1% of at least one vitamin D derivative,
from 10% to 95% of fatty phase,
from 0.1% to 6% of sucroesters,
from 1% to 30% of polyol,
from 0.05% to 3% of hydrophilic gelling agent,
at least 5% of water,
from 0 to 20% of one or more additives.

More preferentially, the composition is in oil-in-water emulsion form and comprises:
from 0.003% to 0.06% of at least one vitamin D derivative chosen from calcitriol, calcipotriol and 4-[6-ethyl-4′-(1-ethyl-1-hydroxypropyl)-2′-propyl-biphenyl-3-yloxymethyl]-2-hydroxymethylphenylmethanol,
   from 10% to 80% of fatty phase, chosen from caprylic/capric triglycerides and mineral oil, alone or as a mixture,
   from 0.1% to 6% of at least one sucroester chosen from sucrose stearate, sucrose laurate and sucrose palmitate,
   from 1% to 30% of glycerol,
   from 0.05% to 3% of hydrophilic gelling agent, chosen from carbomers and polysaccharides,
   at least 5% of water,
   from 0 to 20% of one or more additives.

According to a second preferred embodiment, the composition is in the form of an oil-in-water emulsion and comprises:
from 0.5% to 10% of at least one phenolic derivative, chosen from hydroquinone and rucinol,
from 10% to 95% of fatty phase, chosen from caprylic/capric triglycerides and PPG-15 stearyl ether, alone or as a mixture,
from 0.1% to 6% of at least one sucroester chosen from sucrose stearate, sucrose laurate and sucrose palmitate,
from 1% to 30% of glycerol,
from 0.05% to 3% of hydrophilic gelling agent, chosen from carbomers and polysaccharides,
at least 5% of water,
from 0 to 20% of one or more additives.

A subject of the present invention is also the use of a composition according to the invention for the manufacture of a medicament for treating:
dermatological complaints associated with a keratinization disorder relating to cell differentiation and proliferation, especially for treating common acne, comedones, polymorphs, acne rosacea, nodulocystic acne, acne conglobata, senile acne, and secondary acnes such as solar acne, medication-related acne or occupational acne;
ichthyosis, ichthyosiform conditions, Darier's disease, palmoplantar keratoderma, leukoplakia and leukoplakiform conditions, and cutaneous or mucous (buccal) lichen;
dermatological complaints with an inflammatory immunoallergic component, with or without cell proliferation disorder, and especially cutaneous, mucous or ungual psoriasis, psoriatic rheumatism, cutaneous atopy, such as eczema, or atopic dermatitis, respiratory atopy or gingival hypertrophy,
pathologies caused by Demodex folliculorum and more particularly cutaneous or ophthalmic rosacea,
dermal or epidermal proliferations, whether benign or malignant, and whether of viral origin or otherwise, especially common warts, flat warts and verruciform epidermodysplasia, oral or florid papillomatoses, T lymphoma;
proliferations that may be induced by ultraviolet radiation, especially basal cell and spinal cell epithelioma;
precancerous skin lesions, especially keratoacanthomas;
immune dermatoses, especially lupus erythematosus;
immune bullous diseases;
collagen diseases, especially scleroderma;
dermatological or general complaints with an immunological component;
skin disorders caused by exposure to UV radiation, photo-induced or chronological ageing of the skin, actinic pigmentations and keratosis, or any pathology associated with chronological or actinic ageing, especially xerosis;
sebaceous function disorders, especially the hyperseborrhoea of acne or simple seborrhoea or seborrhoeic dermatitis;
cicatrization disorders or stretchmarks;
pigmentation disorders, such as hyperpigmentation, melasma, hypopigmentation or vitiligo;
fat metabolism complaints, such as obesity, hyperlipidaemia, non-insulin-dependent diabetes or syndrome X,
inflammatory complaints such as arthritis;
cancerous or precancerous conditions;
alopecia of various origins, especially alopecia caused by chemotherapy or radiation;
immune system disorders, such as asthma, type I sugar diabetes, multiple sclerosis, or other selective dysfunctions of the immune system, or
complaints of the cardiovascular system, such as arteriosclerosis or hypertension.

Preferentially, the composition according to the invention containing ivermectin will be used for treating rosacea, and the composition according to the invention containing hydroquinone will be used for treating pigmentation disorders.

In one preferred mode of use of the composition, it will contain a vitamin D derivative and more particularly calcitriol and will be used for the manufacture of a medicament for treating psoriasis or atopic dermatitis.

In a second preferred embodiment of use of the composition, it will contain rucinol and will be used for the manufacture of a medicament for treating pigmentation disorders.

The examples that follow show, in a non-exhaustive manner, examples of formulation of the composition according to the invention and also chemical and physical stability results.

### EXAMPLE 1

### Stability of Calcitriol in Various Solvent Oils

In order to optimize the dissolution of the active agent to be incorporated into the compositions according to the invention, stability tests are performed in various solvent oils:

The preparations are prepared according to the following proportions: 0.00666% of calcitriol/0.29629% of BHT, a sufficient amount of solvent oil to obtain 100%.

| | |
|---|---|
| Solvent | Caprylic/capric triglycerides |

| | | |
|---|---|---|
| % Calcitriol/To | T 1 month | T 3 months |
| RT | 100.3 | 99.9 |

| | |
|---|---|
| Solvent | PPG-15 stearyl ether |

| | |
|---|---|
| % Calcitriol/To | T 1 month |
| RT | 100.00 |

| | |
|---|---|
| Solvent | Octyldodecanol |

| | | |
|---|---|---|
| % Calcitriol/To | T 1 month | T 3 months |
| RT | 98.3 | 95.00 |

| | |
|---|---|
| Solvent | Diisopropyl adipate |

| | | |
|---|---|---|
| % Calcitriol/To | T 1 month | T 3 months |
| RT | 101.5 | 97.2 |

### EXAMPLE 2

### Stability of Hydroquinone in Various Solvent Oils

The preparations are prepared according to the following proportions: 4% of hydroquinone, a sufficient amount of solvent oil to obtain 100%.

| Solvent | Caprylic/capric triglycerides |
|---|---|
| % hydroquinone/To | T 3 months |
| 40° C. | 95.00 |
| Solvent | PPG-15 stearyl ether |
| % Hydroquinone/To | T 3 months |
| 40° C. | 96.00 |
| Solvent | Diisopropyl adipate |
| % Hydroquinone/To | T 3 months |
| | 97.4 |

### EXAMPLE 3

### Stability of Ivermectin in Various Solvent Oils

The preparations are prepared according to the following proportions: 1% of ivermectin, a sufficient amount of solvent oil to obtain 100%.

| Solvent | Diisopropyl adipate |
|---|---|
| % Ivermectin/To | T 1 month |
| 40° C. | 96.7 |
| Solvent | PPG-15 stearyl ether |
| % Ivermectin/To | T 1 month |
| 40° C. | 98.07 |
| Solvent | Phenoxyethanol |
| % Ivermectin/To | T 1 month |
| 40° C. | 98.6 |
| Solvent | Benzyl alcohol |
| % Ivermectin/To | T 1 month |
| 40° C. | 98.6 |

### EXAMPLE 4

### Stability of Calcitriol in Various Solvent Oils

The preparations are prepared according to the following proportions: 5% of rucinol, a sufficient amount of solvent oil to obtain 100%.

| Solvent | Caprylic/capric triglycerides |
|---|---|
| % Rucinol (% LC) | T 1 month |
| 40° C. | 99 |
| Solvent | PPG-15 stearyl ether |
| % Rucinol (% LC) | T 1 month |
| 40° C. | 95 |
| Solvent | PEG-35 castor oil |
| % Rucinol (% LC) | T 1 month |
| 40° C. | 96 |
| Solvent | PEG-8 caprylic/capric triglycerides |
| % Rucinol (% LC) | T 1 month |
| 40° C. | 100 |

Assay technique by HPLC against reference substance.

### EXAMPLE 5

### Composition According to the Invention with a Vitamin D Derivative

| Phases | INCI name | Formulation % |
|---|---|---|
| A | Sucrose laurate | 0.625 |
| A | Sucrose palmitate | 0.625 |
| A | Demineralized water | 1.25 |
| A | Glycerol | 3.75 |
| B | Calcitriol | 0.009 |
| B | BHT | 0.04 |
| B | Caprylic/capric triglycerides | 18.741 |
| B | Methyl paraben | 0.2 |
| C | Demineralized water | 73.30 |
| D | Acrylamide/sodium | 1.5 |
| | acryloydimethyltaurate copolymer | |

Specifications at t0: pH 6.24

Macroscopic aspect: fluid white cream

Microscopic aspect: very fine emulsion

### Physical Stability:

| Stability | Time | | | |
|---|---|---|---|---|
| conditions | T 1 month | T 2 month | T 3 month | T 6 month |
| RT | In | In | In | In |
| | accordance | accordance | accordance | accordance |
| | with the | with the | with the | with the |
| | specifica- | specifica- | specifica- | specifica- |
| | tions | tions | tions | tions |
| 40° C. | In | In | In | In |
| | accordance | accordance | accordance | accordance |
| | with the | with the | with the | with the |
| | specifica- | specifica- | specifica- | specifica- |
| | tions | tions | tions | tions |

### Chemical Stability:

The initial time (T1) is considered as 100%.
[symbol] Calcitriol (9 ppm)

| | |
|---|---|
| Stability | Time |

| conditions | T 1 month | T 2 month | T 3 month | T 6 month |
|---|---|---|---|---|
| RT | — | 100.1 | 96.8 | 98.5 |
| 40° C. | 96.1 | 98.6 | 99.1 | 104.9 |

### EXAMPLE 6

### Composition According to the Invention with a Vitamin D Derivative

| Phases | INCI name | Formulation % |
|---|---|---|
| A | Sucrose laurate | 2.00 |
| A | Sucrose palmitate | 2.00 |
| A | Demineralized water | 4.00 |
| A | Glycerol | 12.00 |
| B | Calcitriol | 0.009 |
| B | BHT | 0.04 |
| B | Caprylic/capric triglycerides | 31.8 |
| B | Mineral oil | 13.50 |
| B | Dimethicone 350 cSt | 100 |
| B | Cyclomethicone 5 | 13.5 |
| B | Methyl paraben | 0.2 |
| C | Demineralized water | 19.551 |
| C | Carbomer | 0.1 |
| D | Sodium hydroxide (1% solution) | 0.3 |

Specifications at t0: pH=5.85

Macroscopic aspect: thick white cream

Microscopic aspect: very fine emulsion

### Physical Stability:

| | |
|---|---|
| Stability | Time |

| conditions | T 1 month | T 2 month | T 3 month |
|---|---|---|---|
| RT | In accordance | In accordance | In accordance |
| | with the | with the | with the |
| | specifications | specifications | specifications |
| 40° C. | In accordance | In accordance | In accordance |
| | with the | with the | with the |
| | specifications | specifications | specifications |

### Chemical Stability:

The initial time (T0) is considered as 100%.
[symbol] Calcitriol (9 ppm)

| Stability | Time | | |
|---|---|---|---|
| conditions | T 1 month | T 2 month | T 3 month |
| RT | 103.7 | 102.8 | 101.9 |
| 40° C. | 101.7 | 100.3 | 97.1 |

### EXAMPLE 7

### Composition with Ivermectin

| Phases | INCI name | Formulation % |
|---|---|---|
| A | Sucrose laurate | 2.5 |
| A | Sucrose stearate | 2.5 |
| A | Demineralized water | 5 |
| A | Glycerol | 15 |
| B | Phenoxyethanol | 2.4242 |
| B | Ivermectin | 1.00 |
| B | Diisopropyl adipate | 18 |
| B | Propyl paraben | 0.3030 |
| B | Caprylic/capric triglycerides | 53.2728 |

Macroscopic aspect: thick white cream

Microscopic aspect: very fine emulsion

### EXAMPLE 8

### Composition with Hydroquinone

| Phases | INCI name | Formulation % |
|---|---|---|
| A | Sucrose laurate | 0.625 |
| A | Sucrose palmitate | 0.625 |
| A | Demineralized water | 1.25 |
| A | Glycerol | 3.75 |
| B | Phenoxyethanol | 0.8 |
| B | Hydroquinone | 1.00 |
| B | Diisopropyl adipate | 18.741 |
| B | Methyl paraben | 0.2 |
| C | Demineralized water | 71.509 |
| D | Acrylamide/sodium | 1.5 |
| | acryloydimethyltaurate copolymer | |

### EXAMPLE 9

### Composition in the Form of a Lotion with Calcitriol

| Phases | INCI name | Formulation % |
|---|---|---|
| A | Sucrose laurate | 0.625 |
| A | Sucrose palmitate | 0.625 |
| A | Demineralized water | 1.25 |
| A | Glycerol | 3.75 |
| B | Calcitriol | 0.06 |
| B | BHT | 0.04 |
| B | Caprylic/capric triglycerides | 18.741 |
| B | Methyl paraben | 0.2 |
| C | Demineralized water | 73.709 |
| D | Acrylamide/sodium | 1 |
| | acryloydimethyltaurate copolymer | |

### EXAMPLE 10

### Composition with Calcitriol

| Phases | INCI name | Formulation % |
|---|---|---|
| A | Decaglycerol monomyristate | 1.8 |
| A | Demineralized water | 1.5 |
| A | Glycerol | 1.5 |
| B | Calcitriol | 0.009 |
| B | BHT | 0.04 |
| B | Caprylic/capric triglycerides | 25.401 |
| B | Phenoxyethanol | 0.8 |
| B | Methyl paraben | 0.2 |
| C | Glycerol | 5 |
| C | Demineralized water | 62.25 |
| D | Acrylamide/sodium | 1.5 |
| | acryloydimethyltaurate copolymer | |

### EXAMPLE 11

### Composition with Calcitriol

| Phases | INCI name | Formulation % |
|---|---|---|
| A | Sucrose laurate | 2.00 |
| A | Sucrose palmitate | 2.00 |
| A | Demineralized water | 4.00 |
| A | Glycerol | 12.00 |
| B | Calcitriol | 0.015 |
| B | BHT | 0.04 |
| B | Caprylic/capric triglycerides | 31.80 |
| B | Mineral oil | 13.50 |
| B | Dimethicone 350 cSt | 13.50 |
| B | Methyl paraben | 0.20 |
| C | Demineralized water | 19.545 |
| D | Carbomer | 0.10 |
| E | 10% sodium hydroxide solution | 0.30 |

### EXAMPLE 12

### Composition with Calcitriol

| Phases | INCI name | Formulation % |
|---|---|---|
| A | Sucrose laurate | 0.825 |
| A | Sucrose stearate | 0.825 |
| A | Demineralized water | 1.65 |
| A | Glycerol | 4.95 |
| B | Calcitriol | 0.009 |
| B | BHT | 0.04 |
| B | Caprylic/capric triglycerides | 13.5 |
| B | Cetostearyl alcohol | 1.98 |
| B | Mineral oil | 8.333 |
| B | Propyl paraben | 0.10 |
| B | Phenoxyethanol | 0.80 |
| C | Demineralized water | 60.30 |
| C | Methyl paraben | 0.20 |
| D | Glycerol | 5 |
| E | Acrylamide/sodium | 1.5 |
| | acryloydimethyltaurate copolymer | |

Specifications at t0: pH=5.43

Macroscopic aspect: fluid white cream

Microscopic aspect: very fine emulsion

### Physical Stability:

Time

| Stability conditions | T 1 month | T 2 month |
|---|---|---|
| RT | In accordance | In accordance |
| | with the | with the |
| | specifications | specifications |
| 40° C. | In accordance | In accordance |
| | with the | with the |
| | specifications | specifications |

### Chemical Stability:

The initial time (T0) is considered as 100%.
[symbol] Calcitriol (9 ppm)

Time

| Stability conditions | T 1 month | T 2 month |
|---|---|---|
| RT | 99.9 | 99.2 |
| 40° C. | 98.2 | 95.2 |

### EXAMPLE 13

### Composition of a Rich Cream with Calcitriol

| Phases | INCI name | Formulation % |
|---|---|---|
| A | Sucrose laurate | 1.51 |
| A | Sucrose stearate | 1.51 |
| A | Demineralized water | 3.03 |
| A | Glycerol | 9.09 |
| B | Calcitriol | 0.009 |
| B | BHT | 0.04 |
| B | Caprylic/capric triglycerides | 25.32 |
| B | Mineral oil | 24.84 |
| B | Propyl paraben | 0.10 |
| B | Phenoxyethanol | 0.80 |
| C | Demineralized water | 19.70 |
| C | Methyl paraben | 0.20 |
| E | Simulgel 600 | 0.20 |

Specifications at t0: pH=6.08

Macroscopic aspect: thick white cream

Microscopic aspect: very fine emulsion

### Physical Stability:

| | Time | | |
|---|---|---|---|
| Stability conditions | T 1 month | T 2 month | T 3 month |
| RT | In accordance | In accordance | In accordance |
| | with the | with the | with the |
| | specifications | specifications | specifications |
| 40° C. | In accordance | In accordance | In accordance |
| | with the | with the | with the |
| | specifications | specifications | specifications |

### Chemical Stability:

The initial time (T0) is considered as 100%.
[symbol] Calcitriol (9 ppm)

| | Time | | |
|---|---|---|---|
| Stability conditions | T 1 month | T 2 month | T 3 month |
| RT | 101.4 | 105.4 | 103.8 |
| 40° C. | 104.7 | 104.0 | 100.4 |

### EXAMPLE 14

### Composition with a Combination of Calcitriol and Clobetasol Propionate

| Phases | INCI name | Formulation % |
|---|---|---|
| A | Sucrose laurate | 0.625 |
| A | Sucrose palmitate | 0.625 |
| A | Demineralized water | 1.25 |
| A | Glycerol | 3.75 |
| B | Calcitriol | 0.009 |
| B | BHT | 0.04 |
| B | Caprylic/capric triglycerides | 18.741 |
| B | Methyl paraben | 0.2 |
| C | Demineralized water | 67.30 |
| C | Clobetasol propionate | 0.05 |
| C | Propylene glycol | 8 |
| D | Acrylamide/sodium | 1.5 |
| | acryloydimethyltaurate copolymer | |

### EXAMPLE 15

### Composition Containing Rucinol

| Phases | INCI name | Formulation % |
|---|---|---|
| A | Sucrose stearate | 0.825 |
| A | Sucrose palmitate | 0.825 |
| A | Demineralized water | 1.65 |
| A | Glycerol | 4.95 |
| B | Rucinol | 1.00 |
| B | Caprylic/capric triglycerides | 23.75 |
| B | Methyl paraben | 0.2 |
| C | Demineralized water | 65.3 |
| D | Acrylamide/sodium | 1.5 |
| | acryloydimethyltaurate copolymer | |

Specifications at t0: pH=5.46

Macroscopic aspect: white cream

Microscopic aspect: very fine emulsion

### EXAMPLE 16

### Composition Containing Rucinol

| Phases | INCI name | Formulation % |
|---|---|---|
| A | Sucrose stearate | 2.5 |
| A | Sucrose palmitate | 2.5 |
| A | Demineralized water | 5.00 |
| A | Glycerol | 15.00 |
| B | Rucinol | 3.00 |
| B | Caprylic/capric triglycerides | 69.97 |

### EXAMPLE 17

### Composition with Ivermectin

| Phases | INCI name | Formulation % |
|---|---|---|
| A | Sucrose laurate | 0.825 |
| A | Sucrose stearate | 0.825 |
| A | Demineralized water | 1.65 |
| A | Glycerol | 4.95 |
| B | Phenoxyethanol | 0.8 |
| B | Ivermectin | 0.33 |
| B | Diisopropyl adipate | 5.94 |
| B | Propyl paraben | 0.10 |
| B | Caprylic/capric triglycerides | 17.58 |
| C | Demineralized water | 65.30 |
| C | Methyl paraben | 0.20 |
| D | Acrylamide/sodium | 1.50 |
| | acryloydimethyltaurate copolymer | |

Macroscopic aspect: thick white cream

Microscopic aspect: very fine emulsion

### EXAMPLE 18

### Composition Containing Rucinol

| Phases | INCI name | Formulation % |
|---|---|---|
| A | Sucrose stearate | 1.00 |
| A | Sucrose palmitate | 2.00 |
| A | Demineralized water | 1.5 |
| A | Glycerol | 9.00 |
| B | Rucinol | 2.5 |
| B | Caprylic/capric triglycerides | 34.00 |
| B | Methyl paraben | 0.2 |
| C | Demineralized water | 48.5 |
| D | Acrylamide/sodium | 1.3 |
| | acryloydimethyltaurate copolymer | |

Specifications at t0: pH=6.15

Macroscopic aspect: white cream

Microscopic aspect: very fine emulsion

### EXAMPLE 19

### Composition Containing Rucinol and a Retinoid

| Phases | INCI name | Formulation % |
|---|---|---|
| A | Sucrose stearate | 0.825 |
| A | Sucrose palmitate | 0.825 |
| A | Demineralized water | 1.65 |
| A | Glycerol | 4.95 |
| B | Rucinol | 1.00 |
| B | Caprylic/capric triglycerides | 23.75 |
| B | Methyl paraben | 0.2 |
| C | Demineralized water | 60.0 |
| C′ | Demineralized water | 5.00 |
| C′ | Adapalene | 0.1 |
| C′ | Poloxamer 124 | 0.2 |
| D | Acrylamide/sodium | 1.5 |
| | acryloydimethyltaurate copolymer | |

### EXAMPLE 20

### Procedure for Preparing the Compositions

heat, in the main vat, the aqueous phase A containing the surfactants and, where appropriate, all or some of the polyols at 75° C. with stirring until dissolution of the surfactants is complete;
heat, in a separate container, the fatty phase B to 75° C. At the end of heating, add the dissolved active agent, heated if necessary to 50° C. in some of the solvent oil;
when the two phases are at a temperature of 75° C., perform emulsification by gradually incorporating phase B into phase A with high shear;
next, leave the emulsion to cool with moderate stirring;
from 40° C., gradually add the rest of the aqueous phase C with moderate stirring;
next, add, where appropriate, the elements of phase D, still with moderate stirring;
optionally and, before phase D, the dispersions of the other active agents (phase C′).

### EXAMPLE 21

### Study of the Release/Penetration of the Active Agents formulated in the Compositions According to the Invention

The study was performed with a composition according to the invention containing calcitriol as active agent.

The object of this study is to evaluate the release and penetration of the active agent calcitriol formulated in a composition according to the invention and to compare the results with those of the active agent formulated in a standard ointment, such as the pomade Silkis®.

Besides good stability of the desired active agent in the composition according to the invention, good release/penetration of the active agent should also be achieved in order to be able to distribute the active agent to its target, in the present case the skin. The purpose of this is to be able to obtain the desired therapeutic effect.

The release/penetration of calcitriol is thus measured in and across the skin, in vivo in micropigs, after a single application of calcitriol, and the concentration/amount of calcitriol in the skin is correlated with the pharmacodynamic response.

Specifically, the object is to evaluate whether the amount of active agent present in the skin as released by the composition according to the invention is sufficient to generate the desired pharmacodynamic activity for a therapeutic effect. To do this, a composition according to the invention containing calcitriol at a concentration of 9 μg/g is compared with the reference composition, the pomade Silkis formulated with 9 μg/g of calcitriol.

For each surface of skin treated, the following individual data were calculated and expressed in μg or as percentages of the applied dose.

The measured parameters are the calcitriol concentrations in:
Stratum corneum
Total skin
Measurement of the pharmacodynamic response (expression of the mRNA of 24-hydroxylase)
Correlation between the calcitriol concentrations in the skin and the intensity of the pharmacodynamic response.

As a percentage of the applied dose

Ointment composition 9 ppm

| | | |
|---|---|---|
| SC | 8.09 | 2.91 |
| Skin | 5.86 | 1.74 |

Composition according to Ex. 14 9 ppm

| | | |
|---|---|---|
| SC | 7.88 | 2.85 |
| Skin | 5.20 | 1.47 |

The results show that the amounts of calcitriol released by the composition according to the invention are significantly of the same order as those released by the ointment. Moreover, a significant increase in the expression of mRNA of 24-hydroxylase was demonstrated, taking into account the vitamin D activity, for the two compositions. This implies that the amounts of active agent delivered by the composition according to the invention are sufficient to give therapeutic activity.

1. A pharmaceutical composition in the form of an oil-in-water emulsion, the composition comprising:
at least one water-sensitive active agent selected from the group consisting of a macrocyclic lactone and a phenolic derivative, the active agent being in a dissolved form and chemically stable in the oily phase,
a lipophilic solvent/oily phase for the active agent, said active agent being dissolved in and chemically stable in the oily phase,
at least one polyol selected from the group consisting of trihydric alcohols, tetrahydric alcohols and hexahydric alcohols,
at least 5% water,
wherein the composition is topical and comprises at least one surfactant selected from the group consisting of a sucroester and a polyglycerol ester.

2-5. (canceled)

6. The composition according to claim 1, wherein the macrocyclic lactone is ivermectin.

7. The composition according to claim 1, wherein the phenolic derivative is rucinol or hydroquinone.

8. The composition according to claim 1, further comprising at least one gelling agent.

9. The composition according to claim 8, wherein the gelling agent is selected from the group consisting of an acrylamide, a carbomer and a polysaccharide.

10. The composition according to claim 1, wherein the lipophilic solvent/oily phase for the active agent comprises at least one fatty substance selected from the group consisting of a capric/caprylic triglyceride and a mineral oil.

11. The composition according to claim 1, wherein the sucroester is selected from the group consisting of sucrose stearate, sucrose laurate, sucrose palmitate, and mixtures thereof.

12. The composition according to claim 1, wherein the polyol is glycerol.

13-14. (canceled)

15. The composition according to claim 1, comprising:
from 0.5% to 10% of at least one phenolic derivative, selected from the group consisting of hydroquinone and rucinol,
from 10% to 95% oily phase, selected from the group consisting of a caprylic/capric triglyceride, and a PPG-15 stearyl ether, and mixtures thereof,
from 0.1% to 6% of at least one sucroester selected from the group consisting of sucrose stearate, sucrose laurate and sucrose palmitate,
from 1% to 30% of glycerol,
from 0.05% to 3% of hydrophilic gelling agent, selected from the group consisting of a carbomer and a polysaccharide,
at least 5% of water, and
from 0 to 20% of one or more additives.

16. A method for treating:
dermatological complaints associated with a keratinization disorder relating to differentiation and to proliferation, especially common acne, comedones, polymorphs, acne rosacea, nodulocystic acne, acne conglobata, senile acne, and secondary acnes such as solar acne, medication-related acne or occupational acne,
ichthyosis, ichthyosiform conditions, Darier's disease, palmoplantar keratoderma, leukoplakia and leukoplakiform conditions, and cutaneous or mucous (buccal) lichen,
dermatological complaints with an inflammatory immunoallergic component, with or without cell proliferation disorder, and especially cutaneous, mucous or ungual psoriasis, psoriatic rheumatism, cutaneous atopy, such as eczema, or atopic dermatitis, respiratory atopy or gingival hypertrophy,
benign or malignant dermal or epidermal proliferations, of viral or non-viral origin, especially common warts, flat warts, verruciform epidermodysplasia, oral or florid papillomatoses, and T lymphoma,
proliferations that may be induced by ultraviolet radiation, especially basal cell and spinal cell epithelioma,
precancerous skin lesions, especially kerato-acanthomas,
immune dermatoses, especially lupus erythematosus,
immune bullous diseases,
collagen diseases, especially scleroderma,
dermatological or general complaints with an immunological component,
skin disorders caused by exposure to UV radiation, photo-induced or chronological ageing of the skin, or actinic pigmentations and keratoses, or any pathology associated with chronological or actinic ageing, especially xerosis,
sebaceous function disorders, especially acne-related hyperseborrhoea, simple seborrhoea or seborrhoeic dermatitis,
cicatrization disorders or stretchmarks,
pigmentation disorders, such as hyperpigmentation, melasma, hypopigmentation or vitiligo,
fat metabolism complaints, such as obesity, hyperlipidaemia, non-insulin-dependent diabetes or syndrome X,
inflammatory complaints such as arthritis,
cancerous or precancerous conditions,
alopecia of various origins, especially alopecia caused by chemotherapy or radiation,
immune system disorders, such as asthma, type I sugar diabetes, multiple sclerosis, or other selective dysfunctions of the immune system, or
cardiovascular system complaints such as arteriosclerosis or hypertension, the method comprising applying to a subject in need of such treatment, a thus effective amount of the composition of claim 1.

17. (canceled)

18. A method for treating pigmentation disorders, the method comprising applying to a subject in need of such treatment, a thus effective amount of the composition of claim 15.

19. The composition according to claim 1, wherein the polyol is the trihydric alcohol glycerol, the tetrahydric alcohol diglycerol and the hexahydric alcohol sorbitol.
